(19) United States
(12) Patent Application Publication
(10) Pub. No.: US 2023/0122281 A1
(43) Pub. Date: Apr. 20, 2023

(54) TNA-BASED PROBE FOR DETECTING AND IMAGING A TARGET miRNA IN LIVING CELLS

(71) Applicant: City University of Hong Kong, Hong Kong (HK)

(72) Inventors: Pik Kwan Peggy LO, Hong Kong (HK); Fei WANG, Dongguan (CN); Ling Sum LIU, Hong Kong (HK)

(21) Appl. No.: 17/902,916

(22) Filed: Sep. 5, 2022

Related U.S. Application Data
(60) Provisional application No. 63/256,640, filed on Oct. 18, 2021.

(51) Int. Cl. C12Q 1/6876; C12Q 1/6841; C12Q 1/6818

(52) U.S. Cl. CPC C12Q 1/6876; C12Q 1/6841; C12Q 1/6818; C12Q 2600/178

(57) ABSTRACT

The present invention provides a TNA-based probe for detecting and imaging a target miRNA in living cells. TNA-based probe is composed of a fluorophore-labeled TNA reporter strand partially hybridizing to a quencher-labeled TNA recognition strand which is designed to be antisense to the target RNA transcript via pair pairing. Upon cellular entry without the need of harmful transfection treatment, the quencher-labeled TNA recognition strand binds to targeted transcript, and these target binding events displace the reporter strand from the quencher, resulting in a discrete “turning-on” of the fluorescence. The extent of fluorescence enhancement is quantifiably related to the target RNA expression level. Additionally, the TNA-based probe shows rapid detection response, excellent selectivity and specificity toward target miRNAs and is able to distinguish the target molecules with 1-2 base mismatches.

### CROSS-REFERENCE OF RELATED APPLICATION

This present application claims the benefit of U.S. Provisional Patent Application No. 63/256,640 filed Oct. 18, 2021, which is incorporated by reference herein in its entirety.

### REFERENCE TO SEQUENCE DISCLOSURE

The sequence listing file under the file name “P2190US01_sequence listing.xml” submitted in ST.26 XML file format with a file size of 26 KB created on Oct. 25, 2022 and filed on Nov. 14, 2022 is incorporated herein by reference.

### FIELD OF THE INVENTION

The present invention relates to threose nucleic acid (TNA)-based probes for detecting and imaging miRNA in a living cell and a method and a kit using the TNA-based probes to detect and image miRNA intracellularly.

### BACKGROUND OF THE INVENTION

MicroRNAs (miRNAs), a class of small non-coding RNA molecules of approximately 21-23 nucleotides in length, function in regulating post-transcriptional gene expression via selective messenger RNAs (mRNAs) silencing. Altered expression level of miRNAs leads to pathological progression because various biological activities, including cell proliferation, differentiation, and apoptosis, are associated with miRNAs. Thus, the expression of certain miRNAs is considered a potential biomarker in the diagnosis and prognosis of diseases, for example, cancer. Conventional techniques for miRNA analysis in homogeneous solutions, such as quantitative reverse transcription-polymerase chain reaction (qRT-PCR), northern blotting, microarrays, and next generation sequencing (NGS), have been widely used. However, these analytical methods still have shortcomings that seriously hamper their practical applications. For instance, complex RNA extraction steps are involved in detecting relative miRNA in bulk samples. The pooling of miRNA from cell lysates makes it incapable of real-time monitoring of target miRNA levels in situ for clinical diagnosis and research laboratories. Particularly, longer time-to-result and large quantity of samples are required for northern blotting with low sensitivity and throughput. Although qRT-PCR exhibits high sensitivity and accuracy, large amounts of designed primers and precise temperature control for amplification are required, which increase its experimental costs and analytical complexity. Moreover, the limitations of microarray and NGS techniques are poor reproducibility and back-end informatics, respectively. Currently, direct imaging of miRNA in living cells is very limited although it can potentially provide a valuable means for identifying cancerous cells and evaluating drug efficacy in real-time. Thus, it is imperative to develop a rapid, convenient, cost-effective and sensitive approach for in situ detection of miRNA expression at the single-cell level.

Due to the flexibility offered by natural nucleic acids, hybridization-based probes that are designed to leverage Watson-Crick base pairing for detecting complementary nucleic acid sequences have recently become hotspots in endogenous miRNA detection research. The recognition nucleic acid strands are usually functionalized with fluorescence resonance energy transfer pairs, fluorescent dye quencher pairs, or intercalator dyes of the thiazole orange family to form a molecular beacon for fluorescence analysis upon target binding. To facilitate signal amplification, recognition nucleic acid strands are further integrated into a large variety of nanomaterials, including nanoparticles, liposomes, polymers, manganese dioxide nanosheet, graphene oxide, quantum dots, and self-assembled DNA nanostructures as intracellular probes for live cells and/or tissues. Nonetheless, challenges remain, such as enzymatic degradation, nuclear sequestration, high false-positive signals, cytotoxicity, and the demand for transfection agents, concerning their potential development into viable diagnosis systems.

To improve the applicability of miRNA analysis, xeno nucleic acids (XNAs), which are chemically modified nucleic acid analogs, such as locked nucleic acid (LNA), peptide nucleic acid (PNA), and 2′ O-methyl RNA (2′ OMe RNA), have been used as building blocks to construct biosensors to detect endogenous RNAs in living cells. Compared with DNA-based probes, these chemically modified nucleic acid analog-based probes exhibit high thermal stability and strong binding affinity and specificity toward target RNAs, resulting in shorter detection time and lower detection limits in the femtomolar scale. However, the exploration of XNA-based RNA detection techniques is still limited. Only miRCURY® LNA® miRNA Detection Probes are commercially available from QIAGEN (USA). Some disadvantages of these LNA-based biosensors result from the several sequence limitations in the synthesis of this nucleic acid analog. In particular, the design of LNA oligomers is constrained by four requirements: (1) sequences of more than four LNA nucleotides must be circumvented; (2) sequences of three or more Cs or Gs must be prevented; (3) the GC content must be restricted between 30% and 60%; and (4) self-complementarity or cross-hybridization must be avoided. These limitations in the sequences of LNA oligomers that can be synthesized and used as capture probes in biosensors can markedly impair the detection of some mutations in genes of interest. Thus, the commercially available LNA-based probes are not high-throughput biosensors with widespread applicability in biotechnology or in a clinical setting.

These above limitations in the sequences of XNA oligomers that can be synthesized and used as capture probes in biosensors can markedly impair the detection of some mutations in genes of interest. Despite XNA possessing high specificity and affinity to RNA and thermal stability, the commercially available XNA-based probes are not high-throughput biosensors with widespread applicability in biotechnology or in a clinical setting. Therefore, the present invention addresses these needs.

### SUMMARY OF THE INVENTION

Accordingly, a first aspect of the present invention provides a threose nucleic acid (TNA)-based probe for detecting and imaging a target miRNA in a living cell includes a fluorophore-labeled TNA sense strand and a quencher-labeled TNA recognition strand which is antisense to a target miRNA transcript via base pairing.

In one embodiment, the fluorophore-labeled TNA reporter strand may be a 3′-Cy3 labeled TNA sense strand and the quencher-labeled TNA strand may be a 2′-black hole quencher 1 (BHQ1) labeled TNA recognition strand.

In one embodiment, the fluorophore-labeled TNA sense strand and the quencher-labeled TNA recognition strand are partially hybridized.

In one embodiment, the fluorophore and the quencher are disposed in close proximity, so that the fluorescence of the fluorophore is quenched by the quencher. Furthermore, the fluorophore-labeled TNA sense strand starts emitting fluorescence when the quencher-labeled TNA recognition strand hybridizes with the target miRNA and displaces from the fluorophore-labeled TNA sense strand.

In one embodiment, the TNA-based probe has nuclease stability, thermal stability, and long-term exceptional storage, and the TNA based probe is highly specific and selective toward the target miRNA and able to distinguish one to two base mismatches of the target miRNA.

In one embodiment, the fluorophore-labeled TNA sense strand and the quencher-labeled TNA recognition strand are hybridized in a molar ratio of 1:1.

In one embodiment, the target miRNA may be a cancer-related miRNA. For example, the target miRNA may be Let-7, miR-7, miR-16, miR-18a, miR-21, miR-31, miR-143, miR-145, mir-155, or miR-191.

In accordance with a second aspect of the present invention, a method of detecting and imaging a target miRNA by using the present TAN-based probe is provided. The method incubating the TNA-based probe with the living cell; and valuating the fluorescence intensity of Cy3.

In one embodiment, the fluorescence intensity can reach to nearly maximum in 10 minutes.

In accordance with a third aspect of the present invention, a kit of detecting and imaging a target miRNA in a living cell is provided. The kit includes the present TNA-based probe.

In one embodiment, the kit further comprises scrambled TNA probes as a negative control.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and advantages of the present invention will become more apparent to those of ordinary skill in the art by describing in detail exemplary embodiments thereof with reference to the accompanying drawings, in which:

FIG. 1 depicts an embodiment of a TNA-based probe for miRNA detection;

FIG. 2 depicts the synthetic scheme and structures of TNA polymer and its intermediates;

FIGS. 3A-3B shows the denaturing PAGE gel analysis of synthesized TNA and DNA strands; FIG. 3A shows the PAGE gel result of synthesized TNA oligonucleotides; and FIG. 3B exhibits the PAGE gel results of synthesized DNA oligonucleotides;

FIGS. 4A-4D show the Cy3 quenching effect in duplexes of TRS and TSS 15, TSS 13 and TSS 11; FIG. 4A depicts the fluorescence intensity of TNA probe with TRS and TSS 15; FIG. 4B shows the fluorescence intensity of TNA probe with TRS and TSS 13; FIG. 4C shows the fluorescence intensity of TNA probe with TRS and TSS 11; and FIG. 4D exhibit the relative Cy3 fluorescence intensity in duplexes of TRS and three TSS strands (11, 13, and 15 bases);

FIGS. 5A-5E show the characterization of designed TNA probes; FIG. 5A depicts the fluorescence spectra of Cy3-labeled TSS, TNA probes before and after incubation of miR-21 target; FIG. 5B shows the fluorescence spectra of scrambled TNA probes before and after incubation of miR-21 targets; FIG. 5C shows the fluorescence intensities change of TNA probes at 564 nm after addition of miR-21 as a function of incubation time; FIG. 5D depicts the native PAGE analysis of strand displacement reaction in TNA probes with electrophoresis performed at 4° C. for 15 h; and FIG. 5E shows the native PAGE analysis of strand displacement reaction in TNA probes with electrophoresis performed at 4° C. for 6 h;

FIGS. 6A-6D exhibits the results of TNA probes for in vitro detection of miR-21; FIG. 6A shows the fluorescence spectra of TNA probes after addition of miR-21 targets at various concentrations; FIG. 6B shows the corresponding titration curve of TNA probes after addition of miR-21 targets at various concentrations; FIG. 6C depicts the fluorescence intensities of TNA probes at 564 nm before (as control) and after incubating of miR-21 targets and three control miRNA mimics, including miR-141, miR-143, and miR-429; and FIG. 6D depicts the fluorescence spectra of TNA probes before (as control) and after incubation with miR-21 targets, and the miR-21 targets with one or two bases mismatched;

FIG. 7 shows the calibration curve of the fluorescence intensity at 564 nm against the miR-21 concentration (n=3);

FIGS. 8A-8F shows the stability evaluation of TNA probes; FIG. 8A depicts the fluorescence intensity of FBS-treated TNA probes before and after addition of miR-21 at designated time intervals; FIG. 8B shows the fluorescence intensity of FBS-treated DNA probes before and after addition of miR-21 at designated time intervals; FIG. 8C depicts the denaturing PAGE analysis of TNA probes against nuclease digestion incubated in FBS assay at various time intervals; FIG. 8D depicts the denaturing PAGE analysis of DNA probes against nuclease digestion incubated in FBS assay at various time intervals; FIG. 8E exhibits the relative fluorescence intensities of detection probes made of DNAs and TNAs at 564 nm as a function of temperatures; and FIG. 8F depicts. The fluorescence intensities of TNA probes at 4° C. as a function of time (day) before and after miR-21 addition;

FIGS. 9A-9B show the normalized area of TNA and DNA probes based on the denaturing PAGE results and corresponding half-life calculation; FIG. 9A shows the normalized area of TNA probes; and FIG. 9B depicts the normalized area of DNA probes; and

FIGS. 10A-10G depict the ability of TNA probes for miR-21 imaging and detection in live cells; FIG. 10A shows the relative cell viability of HEK293 and HeLa cells incubated with TNA duplexes for 24 h determined via MTT assays; FIG. 10B shows CLSM images of HeLa cells incubated with or without TNA probes for 24 h (scale bar=100 μm); FIG. 10C exhibits CLSM images of HeLa cells incubated with TNA probes for different time intervals, and the nucleus is stained with Hoechst 33258 dye (blue, scale bar=100 μm); FIG. 10D exhibits CLSM images of HeLa cells stained with LysoTracker (red) after incubation with TNA probes (green) for various time intervals (scale bar=25 μm); FIG. 10E shows CLSM images of TNA probe treated HEK293 cells and HeLa cells after incubation for 24 h (scale bar=100 μm); FIG. 10F depicts CLSM images of HEK293 and HeLa cells incubated with scrambled and miR-21 targeting TNA probes for 24 h, and nucleus is stained with Hoechst 33258 dye (blue, scale bar=25 μm); and FIG. 10G shows CLSM images of TNA probe-treated HeLa cells (control) and HeLa cells transfected with anti-miR-21, followed by incubation of TNA probes for 24 h (scale bar=75 μm).

### DETAILED DESCRIPTION

In the following description, a threose nucleic acid (TNA)-based probe for detecting and imaging miRNA in a living cell and a method and a kit using the TNA-based probe to detect and image miRNA intracellularly are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, the present invention provides a TNA-based probe for detecting and imaging a target miRNA in living cells. Specifically, the invention uses TNA as a building component to construct a biocompatible probe for rapid and selective fluorescence detection of intracellular RNA targets. The TNA-based probe is composed of a fluorophore-labeled TNA reporter strand partially hybridizing to a quencher-labeled TNA recognition strand which is designed to be antisense to a target RNA transcript via pair pairing. Hybridization of the reporter sequence holds the fluorophore in close proximity to the quencher, effectively quenching its fluorescence. Upon cellular entry without the need of harmful transfection treatment, the quencher-labeled TNA recognition strand binds to a targeted transcript and forms a longer and thermodynamically stable complex. These target binding events displace the reporter strand from the quencher, resulting in a discrete “turning-on” of the fluorescence. The extent of the fluorescence enhancement is quantifiably related to the target RNA expression level. Additionally, the TNA-based probe shows rapid detection response, excellent selectivity and specificity toward target miRNAs and is able to distinguish the target molecules with 1-2 base mismatches. As compared to DNA probes, the superior nuclease and thermal stability in addition to the excellent long-term storage stability make TNA probes biocompatible and cost-effective biosensors for dynamic real-time imaging of target miRNA expression in living cells.

Normally, the target miRNA is a biologically significant miRNA that may relate to metabolic activity, disease progression, cell profile, and other cell biology events. For example, the target miRNA may be a cancer-related miRNA, including Let-7, miR-7, miR-16, miR-18a, miR-21, miR-31, miR-143, miR-145, mir-155, and miR-191.

In accordance with a second aspect of the present invention, a method of detecting and imaging a target miRNA by using the TAN-based probe is provided. By incubating with a living cell, the TNA-based probe can enter into the cell with no cytotoxicity. After the hybridization of the target miRNA and the TNA recognition strand, the TNA sense strand is free and the fluorophore on it can start emitting, which provides a means to evaluate the fluorescence intensity of fluorophore to estimate the concentration of the target miRNA and image the fluorescence display in the living cell.

In accordance with a third aspect of the present invention, a kit for detecting and imaging a target miRNA in a living cell by using the TNA-based probe is provided.

Below the preferred embodiments of the present invention are described in the Examples; it should be appreciated that preferred embodiments described herein are only intended for description and interpretation of the present invention, and not intended to be used as limiting the present invention.

### EXAMPLES

### Example 1

Design of TNA Probes for miRNA Detection

The TNA oligonucleotides used in the present invention are synthesized in accordance with the solid-phase synthetic protocol well known in the art. Briefly, a TNA recognition strand has a sequence of 3′-ATCGAATAGTCTGACTACAACT-BHQ1-2′ (SEQ ID NO. 01), TNA sense strand 15 has a sequence of 3′-Cy3-AGTTGTAGTCAGACT-2′ (SEQ ID NO. 02), TNA sense strand 13 has a sequence of 3′-Cy3-AGTTGTAGTCAGA-2′ (SEQ ID NO. 03), TNA sense strand 11 has a sequence of 3′-Cy3-AGTTGTAGTCA-2′ (SEQ ID NO. 04), TNA scrambled recognition strand has a sequence of 3′-TGAATGCAACGCTCAATACTTA-BHQ1-2′ (SEQ ID NO. 05), TNA scrambled sense strand 15 has a sequence of 3′-Cy3-TAAGTATTGAGCGTT-5′ (SEQ ID NO. 06), DNA recognition strand has a sequence of 5′-BHQ1-TCAACATCAGTCTGATAAGCTA-3′ (SEQ ID NO. 07), DNA sense strand 15 has a sequence of 5′-TCAGACTGATGTTGA-Cy3-3′ (SEQ ID NO. 08), miR-21 (DNA) has a sequence of 5′-TAGCTTATCAGACTGATGTTGA-3′ (SEQ ID NO. 09), miR-21 with one-base mismatch (DNA) has a sequence of 5′-TAACTTATCAGACTGATGTTGA-3′ (SEQ ID NO. 10), miR-21 with two-base mismatch (DNA) has a sequence of 5′-TAACTAATCAGACTGATGTTGA-3′ (SEQ ID NO. 11), anti-miR-21 (DNA) has a sequence of 5′-TCAACATCAGTCTGATAAGCTA-3′ (SEQ ID NO. 12), miR-141 (DNA) has a sequence of 5′-TAACACTGTCTGGTAAAGATGG-3′ (SEQ ID NO. 13), miR-143 (DNA) has a sequence of 5′-TGAGATGAAGCACTGTAGCTC-3′ (SEQ ID NO. 14), miR-429 (DNA) has a sequence of 5′-TAATACTGTCTGGTAAAACCGT-3′ (SEQ ID NO. 15), and miR-21 (RNA) has a sequence of 5′-UAGCUUAUCAGACUGAUGUUGA-3′ (SEQ ID NO. 16).

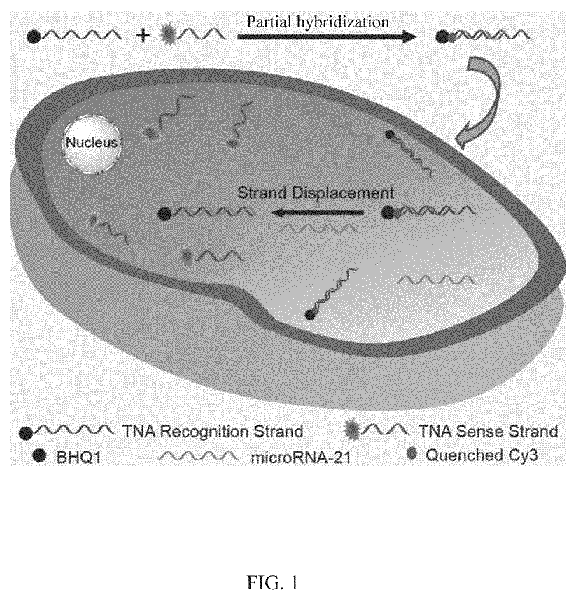

As shown in FIG. 1, the TNA-based probes are constructed by partial hybridization of 2′-black hole quencher 1 (BHQ1)-labeled TNA recognition strand (TRS) with 3′-Cy3-labeled TNA sense strand (TSS). The TRS is designed to be antisense to a target microRNA-21 (miR-21) which is a key regulatory molecule overexpressed in various cancer cells for tumor initiation and progression. In the absence of target miRNAs, the fluorescence signal of Cy3 is very weak and difficult to detect due to the quenching effect of BHQ1, which is in close proximity to Cy3. In the presence of the target miRNA, the Cy3-labeled TTS is displaced by the target molecule and is then dissociated from the BHQ1-labeled TRS. Meanwhile, the TRS is fully hybridized with its complementary target miR-21 to form a thermodynamically stable complex. These binding events separate the BHQ1 and Cy3 fluorophore, resulting in the recovery of Cy3 fluorescence signal. The extent of fluorescence enhancement is quantifiably related to the expression level of the target miRNAs.

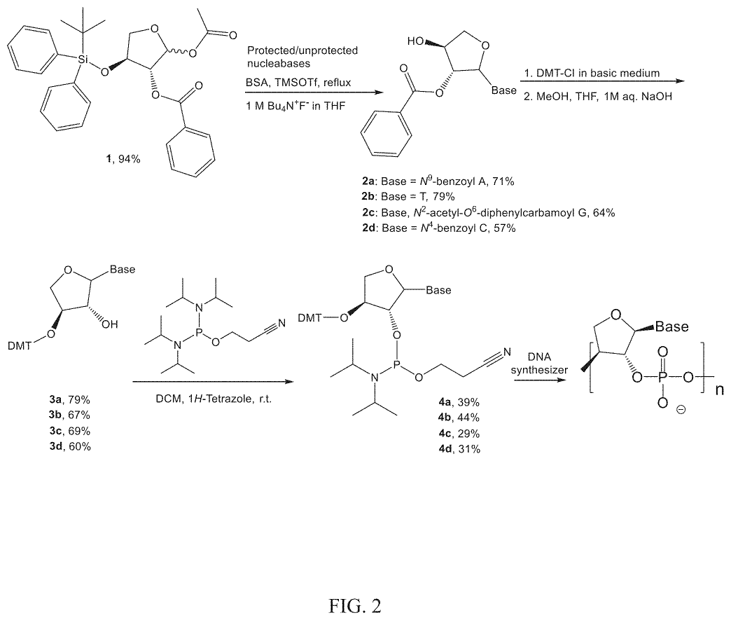

Briefly, TNA oligonucleotides are synthesized according to a reported solid-phase synthetic protocol which involves the standard cyanoethylphosphoramidite chemistry. As shown in FIG. 2, four L-threofuranosyl nucleoside monomers 3a-d are synthesized from 1-O-Acetyl-2-O-benzoyl-3-O-tert-butyldiphenylsilyl-L-threofuranose 1. Four different TNA nucleosides 2 a-d are formed via the Silyl-Hilbert-Johnson reaction by reacting compound 1 with corresponding protected or unprotected nucleobases in the presence of a Lewis acid and then followed by removing the 3′-silyl protecting groups using tetrabutylammonium fluoride. The 3′-OH group on compound 2 is then protected with DMT functional group under basic media and then followed by deprotection of 2′-OH group using sodium hydroxide solution to generate 3a-d. Strategically, 2-cyanoethyl N,N,N,N-tetraisopropylphosphoramidite is used to phosphorylate compounds 3a-d, giving rise to the corresponding 2′-phosphoramidites TNA monomers 4a-d. Sequence-defined TNA polymers are synthesized with the monomers 4a-d on a controlled pore glass (CPG) solid support using an automated nucleic acid synthesizer.

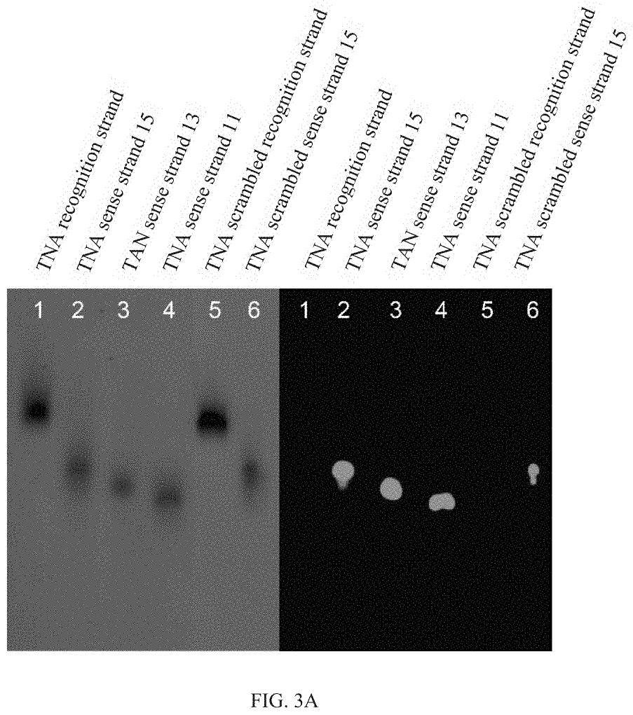

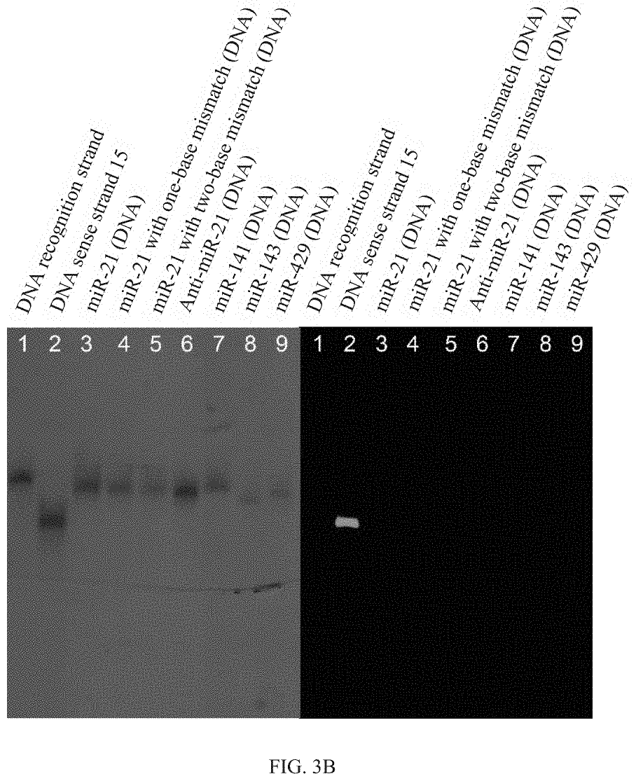

The synthetic TNA and DNA oligonucleotides with designed sequences are confirmed by analysis and denaturing polyacrylamide gel electrophoresis (PAGE) analysis. The PAGE is performed in 1×TBE buffer with a current of 30 mA at room temperature for 1 h. As shown in FIGS. 3A-3B, denaturing PAGE gel analyses confirm the sequences of synthesized TNA and DNA oligonucleotides.

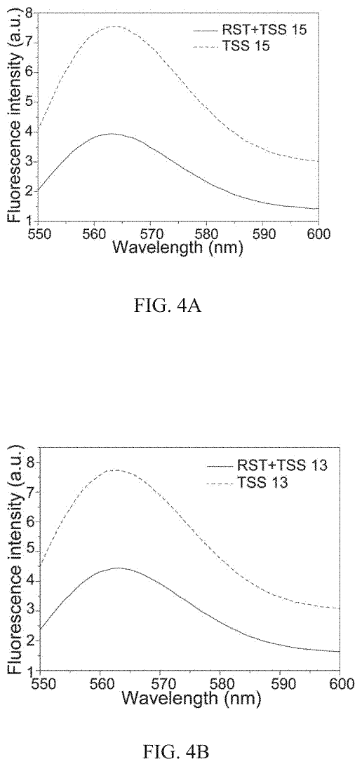

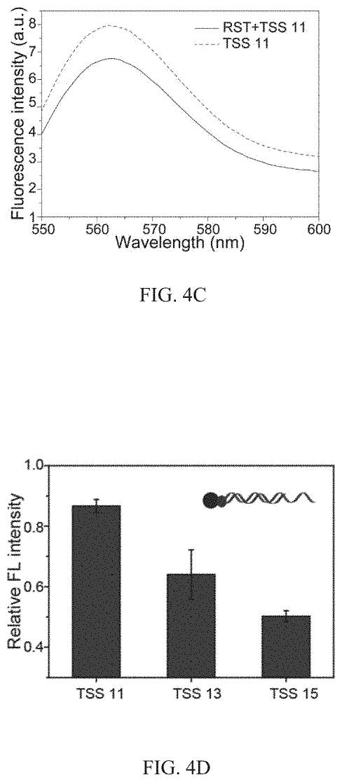

To achieve the maximum quenching effect between Cy3 and BHQ1 before target binding, the fluorescence spectra of the TNA probe having 22-mer TRS and TSS with different number of nucleobases (11-mer, 13-mer, and 15-mer) are measured and compared. In brief, the fluorescence intensity of TNA probes (500 nM) using various Cy3-TSSs (11, 13, and 15 bases) is measured. As shown in FIGS. 4A-4D, the detected fluorescence intensity of TNA probe including TRS and TSS-11, TSS-13 and TSS-15 and the relative intensity indicating that the TNA probe with TRS and TSS-15 has the best quenching effect down to 50%. Thus, TRS and TSS 15 are selected to form the duplex TNA-based probe for following examples. As a control, corresponding TNA duplexes with scrambled sequences were also constructed as scrambled TNA probes.

### Example 2

Target Recognition and Detection

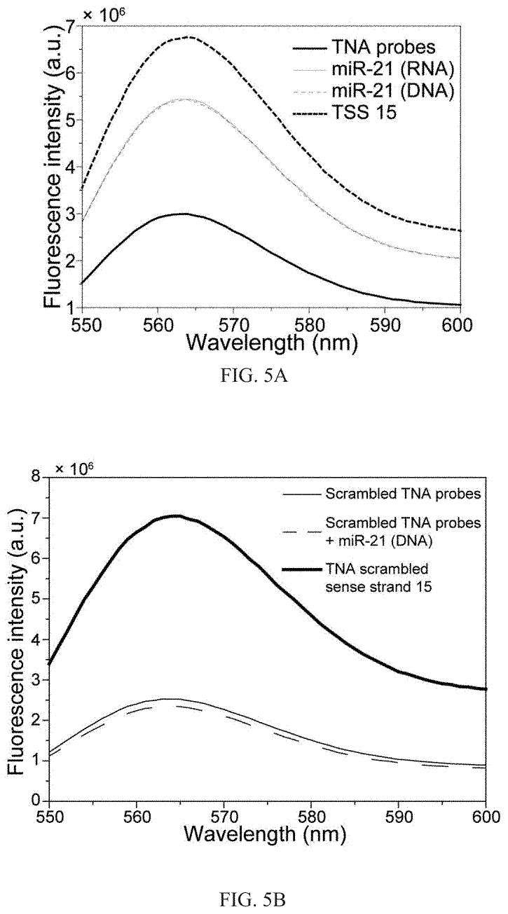

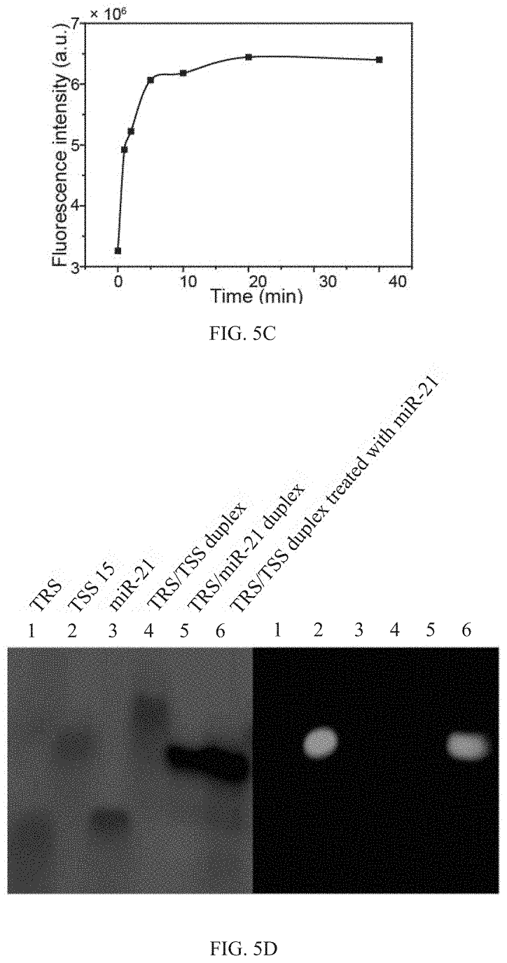

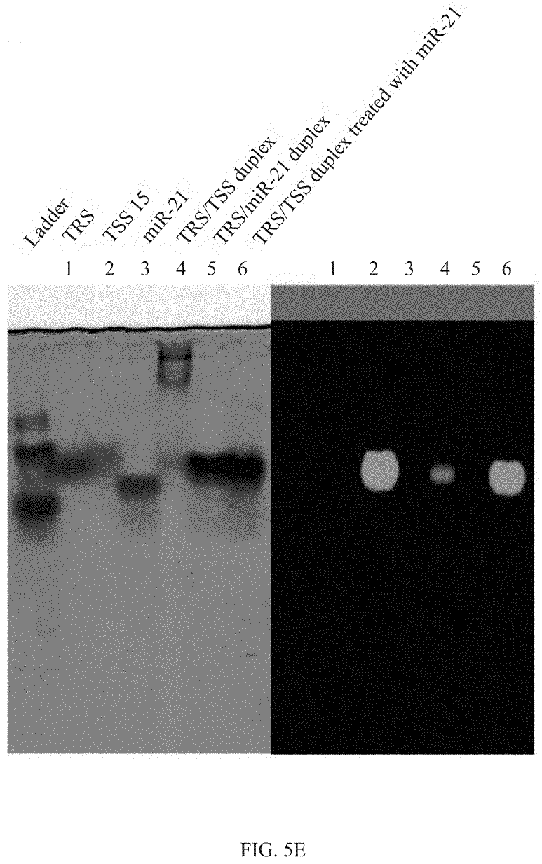

To verify the detection strategy, the fluorescence intensity of duplex TNA-based probes in phosphate-buffered saline (PBS) buffer before and after incubation with the same number of moles of target miR-21 for 40 min at 37° C. is measured. As shown in FIG. 5A, the Cy3 fluorescence signal in solution is increased by 80% after miR-21 is added. In contrast, fluorescence from the scrambled TNA probes incubated with miR-21 is negligible indicating that the strand displacement reaction is sequence dependent. response upon the miR-21 incubation, indicating the strand displacement reaction is sequence-dependent, see FIG. 5B. An DNA or RNA analogs achieved similar fluorescence recovery, strongly suggesting that the sugar conformation of the target molecules does not affect target recognition. Considering the relatively high cost and poor enzymatic resistance of RNA molecules, miR-21 DNA mimics are subsequently used for in vitro detection assays. To further optimize the detection kinetics, the displacement reaction kinetics of the designed TNA probes are evaluated by measuring the Cy3 fluorescence intensities of the TNA probe at different time points after the addition of equivalent moles of target miR-21. The Cy3 fluorescence increases for ˜10 min before reaching a plateau corresponding to the complete strand displacement reaction as shown in FIG. 5C. Native PAGE analysis also confirms the formation of different components after the strand displacement reaction in these binding events. As shown in FIG. 5D, a band in lane 4 with the slowest mobility is observed for the TRS/TSS duplexes (TNA probe). In contrast, after adding miR-21 mimics into the well-formed TRS/TSS duplex probes, a new band is observed in lane 6 with higher mobility, which is equivalent to the TRS/miR-21 complexes in lane 5. In addition, a strong Cy3 signal in lane 6 is observed, which is equivalent to free TSS 15 in lane 2. The diffusion of the bands is due to the prolonged electrophoresis in native PAGE. Referring to FIG. 5E, an additional native PAGE experiment with a shorter running time (6 h) is conducted and the fluorescence recovery is still observed. These results confirmed the formation of TRS/miR-21 duplexes and the dissociation of TSS 15 from TRS during the strand displacement reaction.

### Example 3

Specificity and Sensitivity for Target miRNA Detection

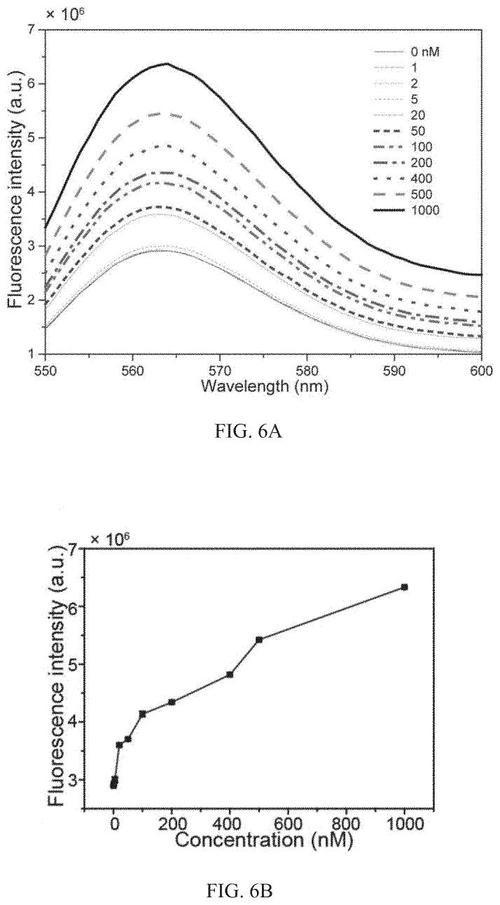

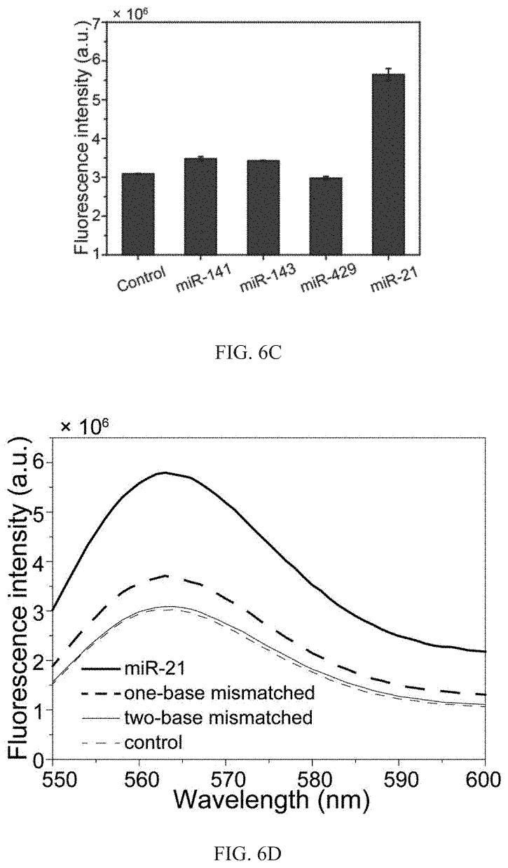

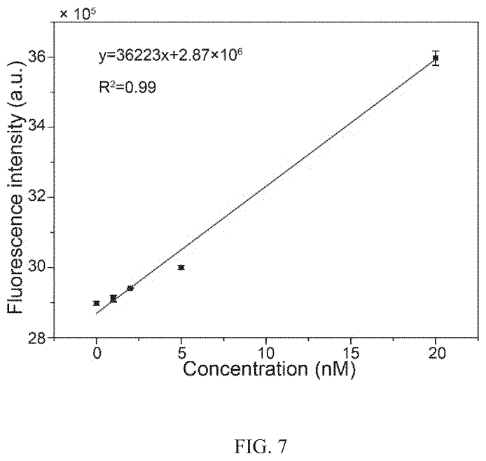

The TNA probe is further evaluated in vitro for its utility in quantifying complementary miRNA targets in a sequence-specific manner. Solutions of duplex TNA-based probes (500 nM) are examined before and after adding 1-1,000 nM miR-21 in PBS buffer for 40 min at 37° C. As indicated in FIG. 6A and FIG. 6B, upon addition of the target RNA, Cy3 fluorescence increases as miR-21 concentrations increase. Therefore, fluorescence activation is correlated with the concentration of target RNAs. In FIG. 7, it shows a linear correlation between target miR-21 (0-20 nM) and fluorescence peak intensities at 564 nm (r²=0.99). The limit of detection (LoD) is calculated to be 0.654 nM, which is comparable with the prior art for miRNA detection.

To investigate the specificity, 500 nM of the control miRNA mimics (miR-141, miR-143, and miR-429) are added into the TNA probes (500 nM) in PBS buffer. Compared with target miR-21, negligible changes of fluorescence intensity at 564 nm are observed, see FIG. 6C. The selectivity of the designed TNA probe is also evaluated by comparing the fluorescence signal generated by the miR-21 target with those containing one or two mismatched sequences. As shown in FIG. 6D, the TNA probes exhibit a distinct difference in fluorescence enhancement toward the target miR-21 and a target miR-21 with one or two base mismatches. This indicates excellent selectivity of the designed TNA probes and their capability to distinguish base mismatches in target RNA molecules.

### Example 4

Biological Stability of Detection Probes Made of TNAs and DNAs

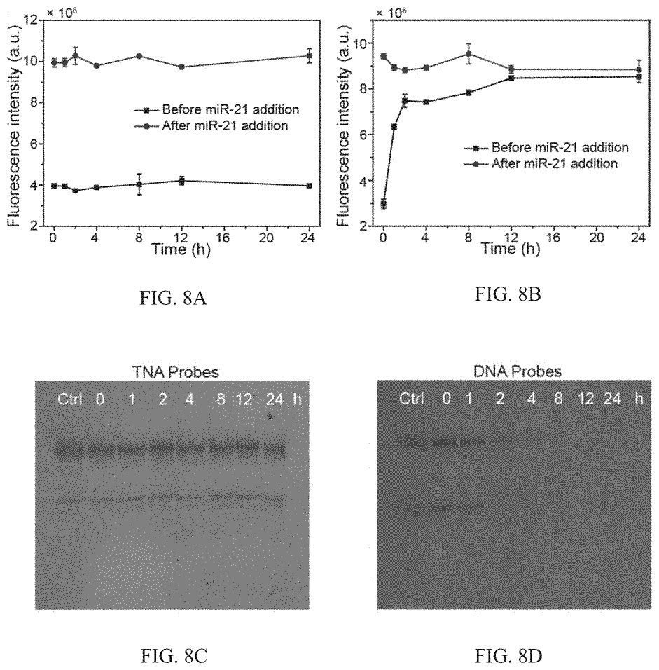

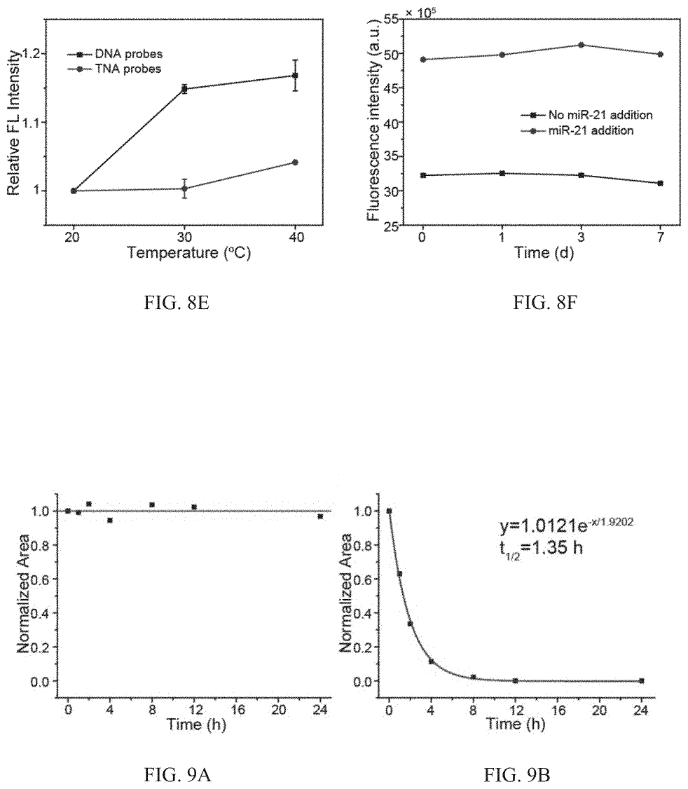

To investigate the stability of TNA, the nuclease stability of TRS/TSS duplexes is investigated to prevent this TNA probe from being destroyed within living cells. The TNA and corresponding DNA probes (1 μM) are incubated separately in PBS buffer containing 10% fetal bovine serum (FBS) at 37° C. for different durations (0, 1, 2, 4, 8, 12, 24 h), and the fluorescence peak intensity at 564 nm is subsequently measured. After the fluorescence intensity measurement at each time point, 500 nM target miR-21 is added into the corresponding samples. After incubation, the fluorescence intensity is measured again. As shown in FIG. 8A and FIG. 8B, the mixture of TNA probe and FBS do not significantly change fluorescence signals at 564 nm as a function of incubation time up to 24 h, indicating that TNA probes are excellent at protecting nuclease cleavage. When target miR-21 is added to the FBS-treated TNA probes, the fluorescence signals are enhanced significantly and at similar intensities at various time points after hybridization proving that the fluorescence recovery is due to the target binding instead of nuclease degradation. These results suggest that the FBS-treated TNA probes are still intact before targets binding. In contrast, the rapid enhancement of fluorescence intensity at 564 nm of DNA probes after 2 h FBS treatment is attributed to the enzymatic degradation of DNA probes prior to target miRNA binding. As shown in FIG. 8C and FIG. 8D, denaturing PAGE analysis shows no degradation of TNA probes after FBS treatment for 24 h when there is a rapid digestion of DNA probes upon FBS treatment with a calculated half-life of 1.35 h. Based on the denaturing PAGE results and corresponding half-tome calculation, the normalized area of TAN probes and DNA probes (FIGS. 9A-9B) further confirms that rapid digestion of DNA probes was observed upon FBS treatment with a calculated half-life of 1.35 h.

The enzymatic resistance assays clearly demonstrate that the DNA-based probes suffer from poor enzymatic resistance and exhibit a false positive signal in physiological conditions. Importantly, the TNA probes can avoid the degradation upon nuclease incubation and maintain the capability of rapid and accurate miRNA sensing.

The influence of temperature change on the TNA probes was also investigated. For the thermal stability, TNA or DNA probes (500 nM) are prepared and incubated at designated temperatures (20° C., 30° C., and 40° C.) for 40 min. After incubation, the fluorescence intensity is measured and analyzed. As shown in FIG. 8E, the TNA probes are insensitive to temperature demonstrated by less variations at fluorescence intensity at 564 nm compared with DNA probes upon temperature elevation.

Furthermore, the long-term storage stability of TNA probes is evaluated by measuring the time-dependent Cy3 fluorescence intensity after incubation at 4° C. TNA probes (500 nM) are prepared in PBS buffer and incubated at 4° C. The fluorescence intensity is monitored at designated time points (0, 1, 3, and 7 days). After the measurement of fluorescence intensity at each time point, target miR-21 is added into the corresponding samples. After incubation, the fluorescence intensity is measure as well. As shown in FIG. 8F, there is negligible change in fluorescence signals when stored for up to 7 days. On the other hand, the TNA probe maintains its capability for target detection with enhanced fluorescence signals upon addition of miR-21, indicating long-term stable duplex hybridization of TNA probes.

### Example 5

miRNA Imaging in Living Cells

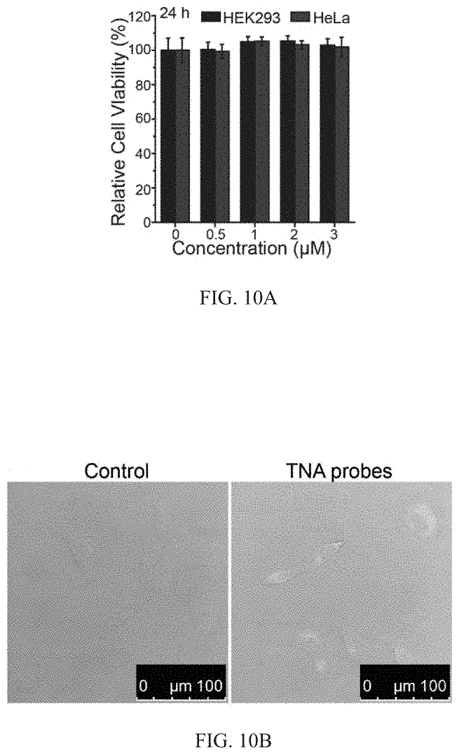

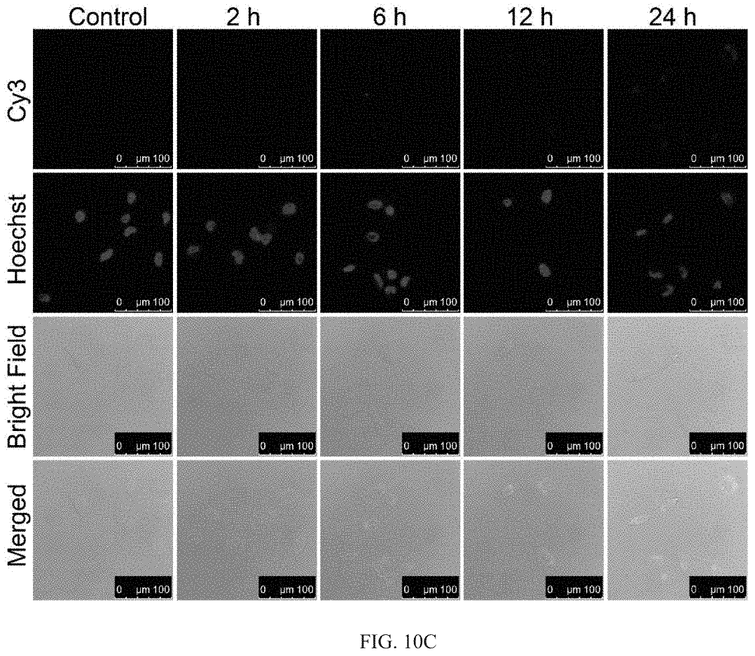

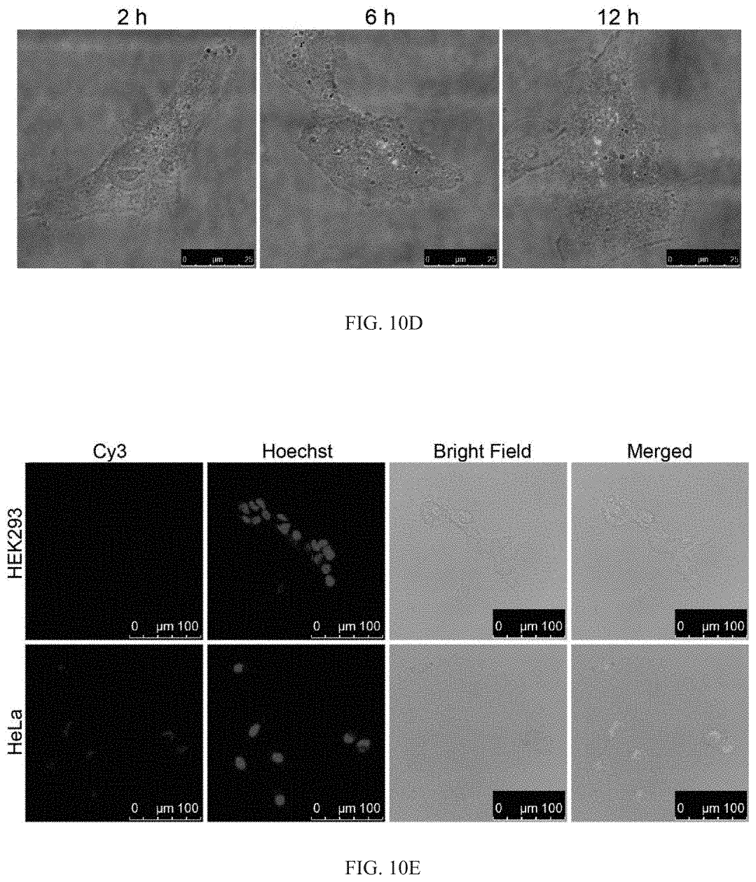

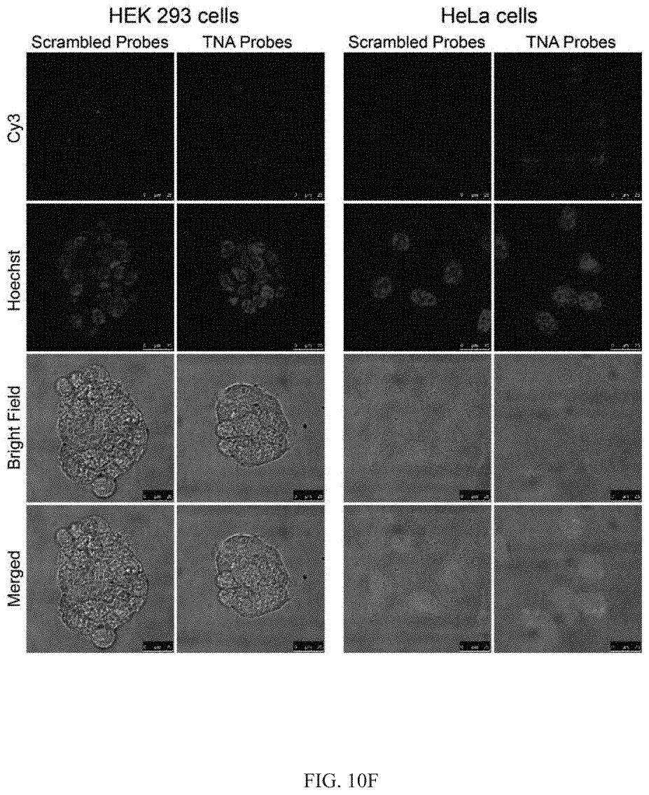

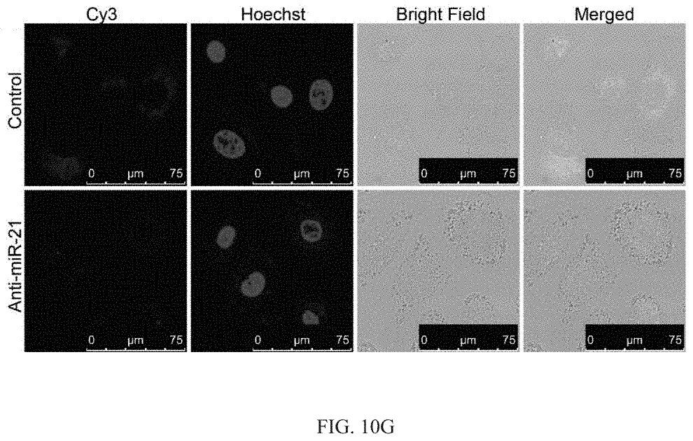

Fluorescence imaging is a real-time, non-invasive, and radiation-free strategy that is widely used in medical diagnosis. TNA oligonucleotides have been reported to internalize and accumulate in cells via a temperature- and energy-dependent endocytic mechanism. Encouraged by the outstanding miRNA detection performance in solution and excellent stability in physiological conditions, the miRNA sensing and imaging by TNA probes in living cells are further investigated. To study the cytotoxicity of TNA duplexes, a standard 3-(4,5-di-methylthiazol-2-yl)-2,5-diphenyltetrazolium bromide (MTT) assay is performed. Briefly, HEK293 cells or HeLa cells are plated in 96-well plates at a density of 10,000 cells per well and incubated overnight at 37° C. Then, the medium is replaced with fresh medium containing TNA-based probes at different concentrations (0, 0.5, 1, 2, and 3 μM); the cells are then incubated for 24 or 48 h. afterward, 20 μL MTT (5 mg/mL) in PBS buffer is added to each well. After incubation for an additional 4 h, the medium is discarded and 150 μL DMSO is added to each well. Plates are incubated at 37° C. for 20 min, and the absorbance at 490 nm is measured on a Bio-Tek Cytation 3 microplate reader. As shown in FIG. 10A, the relative cell viability of HEK293 normal cells and HeLa cancer cells remain at around 100% even after TNA incubation for 48 h, indicating excellent biocompatibility of TNA polymers.

Afterward, the fluorescence response for miR-21 in HeLa cells is investigated and imaged by using confocal laser scanning microscopy (CLSM). Briefly, confocal fluorescence imaging of HeLa cells is set as an example. Typically, HeLa cells are plated in 35-mm glass-bottomed dishes at a density of 150,000 cells per dish and incubated overnight at 37° C. The medium is replaced with medium containing TNA-based probes (100 nM) and cells are further incubated for 24 h. Cells are washed with PBS buffer three times and then fixed with 4% paraformaldehyde at room temperature for 15 min. Fixed cells are then washed with PBS buffer (pH 7.4) for 3 times. After washing with PBS buffer three times, the fixed cells were analyzed with a Leica TCS SP5 laser confocal scanning microscope. The excitation wavelength for the Cy3 dye is 514 nm, and the emission is collected at 550 to 600 nm. As shown in FIGS. 10B-10C, time-dependent confocal fluorescence images show that the intracellular fluorescence signal is observed after 6 h incubation and gradually increased with incubation time, confirming its applicability in the cellular environment for at least 24 h. The time-dependent dynamics of miRNA imaging is also evaluated via colocalization CLSM imaging. As shown in FIG. 10D, no fluorescence signal is detected in the HeLa cells after 2 h incubation because TNAs accumulated inside the endosomes or lysosomes where target miRNAs are absent. The intracellular fluorescence signal gradually increases as the TNA probes escaped from the endosome/lysosome. Since the TNA probes exhibited no degradation in the presence of enzymes, it is confirmed that the intracellular fluorescence signal is due to the responsiveness of TNA probes to the miR-21 target inside the cells.

The capability of TNA probes in the evaluation of the relative expression levels of miR-21 in different cell lines is investigated. The HEK293 cell line with minimal expression of miR-21 is selected as a negative cell line and HeLa cancer cells with high expression of miR-21 are selected as a positive cell line. As depicted in FIG. 10E, differing fluorescence signal brightness is observed in the two different cell lines. HeLa cells have much higher fluorescence signals compared with HEK293 cells after incubation with miR-21-targeting TNA probes (in red color), indicating the upregulated expression of miR-21 in HeLa cells. These results are consistent with the previously reported varying expression levels of miR-21 in these two cell lines. An additional confocal fluorescence imaging experiment in which the HeLa and HEK294 cells are incubated with scrambled TNA probes is conducted. As shown in FIG. 10F, HEK293 cells show insignificant differences upon the incubation of scrambled probes and miR-21 targeting TNA probes. HeLa cells that are incubated with miR-21-targeting TNA probes display remarkably enhanced fluorescence intensity compared with cells incubated with scrambled probes. It confirms that the differing response of HEK293 and Hela cells to the developed TNA probes is due to the differential expression of miR-21.

Furthermore, the potential of TNA probes to detect the dynamic change of miR-21 expression level in cells is further investigated. HeLa cells are first transfected with anti-miR-21 to suppress miR-21 expression level and then incubated with TNA probes for imaging. Briefly, HeLa cells are plated in 35-mm glass-bottomed dishes at a density of 100,000 cells per dish. After incubation overnight at 37° C., anti-miR-21 strands are transfected into cells in serum-free medium using Lipofectamine 3000 reagent (Invitrogen, Thermo Fisher Scientific) under the instructions to downregulate the miR-21 expression. As shown in FIG. 13G, very weak fluorescence signals are observed in the TNA probe-treated HeLa cells after anti-miR-21 transfection compared with non-transfected control cells. It shows that the TNA probes can sense and quantitatively image dynamic expression of tumor-related miRNAs in cancer cell lines and can be utilized for differentiation of different cell lines.

In summary, the present invention provides a TNA-based probe for rapid and accurate miRNA detection and imaging in living cells. It shows high binding specificity and affinity toward the target/complementary RNA sequences followed with a strand displacement reaction of the probe for emitting fluorescence signal. The TNA probe not only exhibit insignificant responses to non-target miRNAs, but also are able to distinguish target miRNAs with one to two base mismatches. Compared with DNA probe, the TNA probe of present invention has good nuclease stability, thermal stability, and exceptional storage ability for long-term cellular studies. In addition, TNA probe is efficiently taken up by living cells with negligible cytotoxicity for dynamic real-time monitoring of target miRNAs. Importantly, the TNA probe differentiates the distinct target miRNA expression levels in various cancer cell lines.

1. A threose nucleic acid (TNA)-based probe for detecting and imaging a target miRNA in a living cell, comprising:
a fluorophore-labeled TNA sense strand; and
a quencher-labeled TNA recognition strand, wherein the quencher-labeled TNA recognition strand is antisense to the target miRNA transcript via base pairing.

2. The TNA-based probe of claim 2, wherein the fluorophore-labeled TNA reporter strand is a 3′-Cy3 labeled TNA sense strand.

3. The TNA-based probe of claim 3, wherein the quencher-labeled TNA strand is a 2′-black hole quencher 1 (BHQ1) labeled TNA recognition strand.

4. The TNA-based probe of claim 1, wherein a TNA sense strand and a TNA recognition strand are hybridized in a molar ratio of 1:1.

5. The TNA-based probe of claim 1, wherein the TNA sense strand and a TNA recognition strand are partially hybridized.

6. The TNA-based probe of claim 5, wherein the fluorophore and the quencher are disposed in close proximity for quenching the fluorescence of the fluorophore-labeled TNA sense strand.

7. The TNA-based probe of claim 5, wherein the fluorophore-labeled TNA sense strand starts emitting fluorescence when the quencher-labeled TNA recognition strand hybridizes with the target miRNA and displaces from the fluorophore-labeled TNA sense strand.

8. The TNA-based probe of claim 7, wherein the intensity of the emitted fluorescence quantifiably relates to the target miRNA expression level.

9. The TNA-based probe of claim 1, wherein the target miRNA is a cancer-related miRNA, comprising Let-7, miR-7, miR-16, miR-18a, miR-21, miR-31, miR-143, miR-145, mir-155, and miR-191.

10. A method of detecting and imaging a target miRNA in a living cell by using the TNA-based probe of claim 1, comprising:
incubating the TNA-based probe with the living cell; and
evaluating the fluorescence intensity of Cy3.

11. The method of claim 10, wherein the fluorescence intensity can reach to its maximum in 10 minutes.

12. A kit of detecting and imaging a target miRNA in a living cell, comprising the TNA-based probe of claim 1.

13. The kit of claim 12, wherein the kit further comprises a scrambled TNA probe as a negative control.


SEQUENCE LISTING (16 sequences)

SEQ ID NO: 1 - DNA, 22 bp, synthetic construct
  source 1..22: mol_type = other DNA
  modified_base 1..22: mod_base = OTHER; note = alpha- (L) - threofuranosyl - (3'-2') nucleic acid TNA
atcgaatagtctgactacaact

SEQ ID NO: 2 - DNA, 15 bp, synthetic construct
  source 1..15: mol_type = other DNA
  modified_base 1..15: mod_base = OTHER; note = alpha- (L) - threofuranosyl - (3'-2') nucleic acid TNA
agttgtagtcagact

SEQ ID NO: 3 - DNA, 13 bp, synthetic construct
  source 1..13: mol_type = other DNA
  modified_base 1..13: mod_base = OTHER; note = alpha- (L) - threofuranosyl - (3'-2') nucleic acid TNA
agttgtagtcaga

SEQ ID NO: 4 - DNA, 11 bp, synthetic construct
  source 1..11: mol_type = other DNA
  modified_base 1..11: mod_base = OTHER; note = alpha- (L) - threofuranosyl - (3'-2') nucleic acid TNA
agttgtagtca

SEQ ID NO: 5 - DNA, 22 bp, synthetic construct
  source 1..22: mol_type = other DNA
  modified_base 1..22: mod_base = OTHER; note = alpha- (L) - threofuranosyl - (3'-2') nucleic acid TNA
tgaatgcaacgctcaatactta

SEQ ID NO: 6 - DNA, 15 bp, synthetic construct
  source 1..15: mol_type = other DNA
  modified_base 1..15: mod_base = OTHER; note = alpha- (L) - threofuranosyl - (3'-2') nucleic acid TNA
taagtattgagcgtt

SEQ ID NO: 7 - DNA, 22 bp, synthetic construct
  source 1..22: mol_type = other DNA
tcaacatcagtctgataagcta

SEQ ID NO: 8 - DNA, 15 bp, synthetic construct
  source 1..15: mol_type = other DNA
tcagactgatgttga

SEQ ID NO: 9 - DNA, 22 bp, synthetic construct
  source 1..22: mol_type = other DNA
tagcttatcagactgatgttga

SEQ ID NO: 10 - DNA, 22 bp, synthetic construct
  source 1..22: mol_type = other DNA
taacttatcagactgatgttga

SEQ ID NO: 11 - DNA, 22 bp, synthetic construct
  source 1..22: mol_type = other DNA
taactaatcagactgatgttga

SEQ ID NO: 12 - DNA, 22 bp, synthetic construct
  source 1..22: mol_type = other DNA
tcaacatcagtctgataagcta

SEQ ID NO: 13 - DNA, 22 bp, synthetic construct
  source 1..22: mol_type = other DNA
taacactgtctggtaaagatgg

SEQ ID NO: 14 - DNA, 21 bp, synthetic construct
  source 1..21: mol_type = other DNA
tgagatgaagcactgtagctc

SEQ ID NO: 15 - DNA, 22 bp, synthetic construct
  source 1..22: mol_type = other DNA
taatactgtctggtaaaaccgt

SEQ ID NO: 16 - RNA, 22 nt, synthetic construct
  source 1..22: mol_type = other RNA; note = the symbol"t" stands for uracil in RNA
tagcttatcagactgatgttga